(19) United States
(12) Patent Application Publication
(10) Pub. No.: US 2019/0142890 A1
(43) Pub. Date: May 16, 2019

(54) DIETARY SUPPLEMENT DERIVED FROM THE SENEGALIA PLANT

(71) Applicant: Hi-Tech Pharmaceuticals, Inc., Norcross, GA (US)

(72) Inventor: Jared Wheat, Alpharetta, GA (US)

(73) Assignee: Hi-Tech Pharmaceuticals, Inc., Norcross, GA (US)

(21) Appl. No.: 15/814,594

(22) Filed: Nov. 16, 2017

(51) Int. Cl. A61K 36/48; A61K 31/522; A23L 33/105

(52) U.S. Cl. CPC A61K 36/48; A61K 31/522; A23V 2250/2108; A23V 2002/00; A23V 2200/332; A23L 33/105

(57) ABSTRACT

A dietary supplement is provided whereby the daily administration of materials derived from the Senegalia plants is provided orally to humans for the purpose of producing or maintaining weight loss as well as improving a person's physical performance and increasing the person's lean muscle mass. The Senegalia materials include these portions of the plant that are normally considered waste or inedible, such as leaves, bark, and roots. The materials contain at least one of the alkaloids from the group consisting of N-methyl-β-phenethylamine; N,N-dimethylphenethylamine; and N-methyl-β-methylphenethylamine. The materials can be administered in their natural form or as extracts, and can be administered in various ways including powders, liquids, capsules and tablets. The Senegalia material may also be used as a tea. For weight loss and weight control, the materials may also be administered concurrently with caloric restriction or in the absence of caloric restriction. The materials may also be administered for the purpose of increasing muscle mass concurrently with a high protein diet as well as an exercise program. More particularly, the present invention relates to the discovery that useful and exploitable levels of these adrenergic amines occur in the plant species of Senegalia.

### FIELD OF THE INVENTION

The present invention relates generally to dietary supplements. In particular, the present invention relates to the use of Senegalia Berlandieri extract alone or in combination with one or more ingredients to control body weight by promoting fat loss, increased energy, suppressing appetite, and/or stimulating thermogenesis. More particularly, the invention relates to the dietary supplement for accomplishing these tasks.

### BACKGROUND OF INVENTION

Losing weight is very difficult for many individuals. Weight gain results when an individual's caloric intake exceeds the number of calories expended as energy. In attempting to lose weight, individuals often strive to attain a caloric deficit (i.e., decreasing caloric intake so that calories expended as energy exceed caloric intake). Generally, this results in an adaptive response of lowered basal (resting) metabolic rate and skeletal muscle loss, which can make it harder to keep the weight off once the individual has attained his desired weight goal.

One cause of the decrease in resting energy expenditure may be reduced functionality of the sympathetic nervous system (“SNS”) of the body. It has been observed that genetically obese rats exhibit low sympathetic outflow or responsiveness in various tissues. Humans genetically predisposed to weight gain already exhibit reduced SNS functioning; decreased energy expenditure further exacerbates their condition.

Physiologically, reduced SNS functioning translates into reduced adrenaline (norepinephrine and epinephrine), and induced thermogenesis (i.e., reduced heat production within the body). Norepinephrine and epinephrine regulate metabolism via stimulation of beta-adrenergic receptors within the SNS. Beta-adrenergic receptors are involved in the pathways of lipolysis, glycogenolysis, and thermogenesis. Thus, reducing SNS functioning impedes these pathways.

Indirect sympathomimetic compounds are adrenergic agents that potentiate the release of norepinephrine and epinephrine at pre-synaptic sites in the SNS and thereby avert SNS functioning resulting from weight loss or reduced caloric intake. In the past, indirect sympathomimetic drugs such as ephedrine have been administered to humans as slimming agents, often in combination with methylxanthines (stimulatory agents) such as caffeine or theophylline. While individuals undergoing such treatment may lose weight, treated individuals may also experience undesirable side effects such as nervousness, tachycardia, hypertension, insomnia, and dry mouth.

The sensation of hunger and fullness (satiety) are regulated by a complex array of interactions between neurochemicals in specific structures of the brain. The most important area in the control of eating behavior is the hypothalamus especially the ventromedial area known as the paraventricular nucleus. Imbedded in the ventromedial area of the hypothalamus are the “feeding center” and the “satiety center,” which are influenced by dietary substances (such as glucose) in the blood and by the neurotransmitters norepinephrine, serotonin and dopamine. These two centers tend to balance each other so that we want to eat but then feel a sense of satisfaction so that we stop eating. This balance can be thrown off by stress or emotional events and changes in certain hormone levels in the blood.

Norepinephrine is a natural “amine” that acts mostly at alpha-receptors in the brain, and directly or indirectly is believed to alter the hypothalamic centers in such a way as to reduce the desire for food. Serotonin and dopamine are also believed to alter eating behavior. Certain synthetic or naturally-occurring chemicals related to norepinephrine, called “sympathomimetic amines,” also have the effect of reducing food intake. This is the basis for the action of prescription drugs like phentermine and Meridia. Ephedrine (from Ephedra) is a natural sympathomimetic amine that causes the release of norepinephrine in the brain. This indirect effect contributes to its efficacy in weight-control products. The possible negative effects of ephedrine have led to the search of alternative natural and effective substances to control the desire to eat.

One alternative is a plant source of natural norepinephrine-like substance called phenethylamine. This sympathomimetic amine is found in the leaves of several species of the plants. Phenethylamine also occurs in cabbage, kale, cauliflower and chocolate. Phenethylamine acts on alpha-receptors in the brain, as do norepinephrine and certain prescription anti-obesity drugs. It is also believed to cause the release of dopamine in the pleasure sensing areas of the brain. However, the action of phenethylamine is rapidly terminated by enzymes in the intestinal wall and liver.

It has long been known that natural and synthetic substances may facilitate weight loss in those who are overweight or obese. Such substances have found utility in this respect may act by a variety of mechanisms. For example, some such substances act by mimicking the effects of endogenous neurotransmitters and are capable of directly replacing these neurotransmitters in their actions on receptors. This, in turn, leads to increased activity of the cells which possess the receptors. Where the receptors concerned are normally responsive to the endogenous hormones adrenaline (epinephrine) and noradrenaline (norepinephrine), which meditate the activities of the sympathetic nervous system, such substances are termed direct-acting sympathicomimetic agents. Typical examples are the amphetamines. Other substances that produce similar effects on the SNS do so by stimulating the release of the endogenous hormones adrenaline and noradrenaline and are thus termed indirect-acting sympathicomimetic agents. Ephedrine is a typical example of an indirect-acting sympathicomimetic agent. Such substances may also be referred to as agonists.

While the formal distinction between direct-acting and indirect-acting sympathicomimetic action is clear, it is realized that many substances which act by causing sympathetic stimulation do so by both mechanisms, depending on intake levels and the receptors involved. Thus, amphetamines act mainly directly, but also have some indirect actions, while ephedrine acts indirectly; but, if given in higher dosage, may also stimulate receptors directly, particularly in the brain. It has been demonstrated that the main perceived actions of sympathicomimetic agents depends both on their differing specificities for the various receptors and on the pharmacokinetic behaviors of the agents in the body.

Thus, the amphetamines, which are direct agents and readily cross the blood-brain barrier, mainly cause central nervous system stimulation, while ephedrine, and particularly pseudoephedrine, are indirect agents which do not cross blood-brain barrier so readily and thus are mainly seen to exert peripheral effects.

Another class of substance of value in assisting weight loss modulates other neurotransmitters, namely, those involved in serotoninergic systems and particularly 5-hydroxytryptamine (“5-HT” otherwise known as serotonin). These substances; of which fenfluramine and its optical isomer, dexfenfluramine, are typical; act by preventing the re-uptake of serotonin into storage granules in neurones. Levels of 5-HT in the synaptic gap thus remain elevated for longer periods, exciting receptors on responsive cells to greater activity.

Other aids to weight loss have been proposed, such as substances which prevent the absorption of nutrients from the digestive system, but the value of such approaches is minimal. Generally, the accepted substances of value in weight loss act by modulating neurotransmitter function in the central nervous system or peripherally.

Substances which modulate neurotransmitter function in the central nervous system are known to act by increasing the availability of catecholamines, in particular, noradrenaline, in certain areas of the brain, thus resulting in perceived suppression of hunger. By suppressing hunger, less food is eaten, and caloric intake is lowered. Examples of such substances include phenylpropanolamine, phentermine, and the amphetamines. Substances which act by increasing the availability of 5-HT, on the other hand, are known to increase perceptions of satiety. An example of such substance is xfenfluramine.

Irrespective of mechanisms, substances of either of these types result in reduced food intake. But their use can be attended by various unwanted effects characteristic of interference with other hormone-regulated systems in the body. It has furthermore been noted that the effects of these types of substances are transient, requiring progressively greater dosage to elicit desired effects until the body finally becomes unresponsive. This progressive decrease in sensitivity is termed tachyphylaxis.

More recently, attention has been focused in ephedrine, which was originally thought to suppress the hunger center in the brain. However, during the last 30 years, research has shown that ephedrine acts mainly by stimulating thermogenesis, increasing the metabolic rate, and stimulating lipolysis.

From the foregoing, it will be obvious to those skilled in the art that the agents most suitable for inducing weight loss in those with excess weight, or, for persons of normal weight, increasing energy availability and/or muscle mass, would be sympathicomimetic (adrenergic) agents whose mechanism of action is mainly indirect, resembling that of ephedrine, and whose pharmacokinetics favor retention of the agents in the periphery rather than passage into the brain. Agents whose profiles match these requirements would be less likely to cause central nervous system stimulation under normal conditions of use, but would still possess enough central action to suppress the hunger center. The partition in favor of peripheral tissues would result in increased levels of these agents at the sites of the beta-3-receptors, which mediate lipolysis and thermogenesis. It is also widely believed that sympathicomimetic agents possessing mainly an indirect mechanism of action would be less likely to cause unwanted side effects and less likely to result in addictive situations. In further detail, the composition of the invention has utility in regulating or treating weight problems as well as increasing vitality, energizing, and in the long term increasing lean muscle mass.

Hitherto, the only such agent which has been shown to act in the optimized ideal fashion has been ephedrine itself. Ephedrine has some drawbacks, however. It is primarily provided in pharmaceutical forms which allow quick release in the body for the alleviation of acute respiratory ailments whereas, for the purpose of inducing lipolysis and thermogenesis, a slower release is desirable. Furthermore, many of those who are overweight prefer not use agents which are presented as drugs. In addition, for a variety of health conditions, such use will often be contraindicated because of the risk of potentially hazardous side effects, which risk could be increased because of the weight problem.

Prior to this invention, those wishing to avail themselves of natural products for eliciting weight loss or increasing muscle mass have had no choice other than to use products containing Ephedra herb (Ephedraceae), which contains ephedrine together with related alkaloids. However, because of concerns about the use of Ephedra herb products, many do not avail themselves of this opportunity.

The provision of a natural product that acts in the ideal fashion noted above would therefore provide major benefits to those seeking to lose weight or improve their physical fitness, or both, and would be especially useful to those who prefer not to take either drug-like products or natural products containing ephedrine alkaloids.

Thus, there exists a need in the art for a safe, effective dietary supplement that promotes weight loss and maintains lean body mass, while avoiding potential negative side effects associated with other dietary supplements used to promote weight loss.

### SUMMARY OF THE INVENTION

The present disclosure is directed to a dietary supplement which promotes weight loss, increases energy, suppresses appetite, and/or stimulates thermogenesis. This is accomplished by ingesting Senegalia Berlandieri extract, or a pharmaceutically acceptable salt thereof alone or combining Senegalia Berlandieri extract or a pharmaceutically acceptable salt thereof with one or more other ingredients designed to modify the body's metabolism.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein.

The present invention discloses the use of adrenergic amines of the group comprising of N-methyl-β-phenethylamine; N,N-dimethylphenethylamine; and N-methyl-β-methylphenethylamine that are useful to assist in weight loss, adding muscle mass, and/or increasing physical performance. The present invention also discloses the discovery that useful and exploitable levels of these adrenergic amines occur in plant species of Senegalia, specifically, Senegalia Berlandieri. Useful levels of these substances occur in the parts of the plant that are not normally eaten, including the leaves, bark, and roots, and can be extracted using methods well known to those of skill in the art.

Provided therefore is a method for promoting energy and weight loss while stimulating thermogenesis and suppressing appetite. The method includes administering to a human a composition including about 0.025 milligrams to about 2.5 milligrams per 1 kilogram of the human's bodyweight of an extract of one of the group consisting of Senegalia Berlandieri.

In one embodiment, the composition is in an oral dosage form. In another embodiment, the oral dosage form is selected from the group consisting of a chewable tablet, a quick dissolve tablet, an effervescent tablet, a hard gelatin capsule, a soft gelatin capsule, a powder, a reconstitutable powder, a suspension, an elixor, a caplet, a health bar, a liquid, a food and combinations thereof. In another embodiment, the oral dosage is selected from the group consisting of immediate release, extended release, pulsed release, delayed release, timed release, variable release, controlled release and combinations thereof. In another embodiment, the composition is administered once during a twenty-four hour period of time. In yet another embodiment, the composition is administered at least twice during a twenty-four hour period of time.

In another embodiment the present invention includes a method for promoting weight loss while stimulating thermogenesis and suppressing appetite which included administering to a human a composition including about 25 milligrams to about 250 milligrams of an active ingredient selected from one of the group consisting of N-methyl-β-phenethylamine; N,N-dimethylphenethylamine; and N-methyl-β-methylphenethylamine, wherein said active ingredient is extracted from Senegalia Berlandieri.

These substances stimulate beta-receptors and release norepinephrine thereby stimulating thermogenesis, increasing metabolic rate, increase energy, and stimulating lipolysis. Appetite and satiety are also regulated by these substances. For the present invention, the amount of adrenergic amines needed to be effective can be as low as 25 milligrams (mg) ingested daily. The preferred use, however, is to administer single doses of from 25 to 250 mg up to three times daily, making a total daily dose of about 75 to 750 mg per day.

In another embodiment, the present invention includes a pharmaceutical combination, which includes an extract of one of the group consisting of Senegalia Berlandieri, or a pharmaceutically acceptable salt thereof and a stimulant. In another embodiment the stimulant is caffeine.

The present invention provides a composition containing an effective amount of at least one of these adrenergic amines to stimulate the addition of lean muscle mass, enhance physical performance, promote weight control, and to stimulate weight loss. The composition can be administered in a form with the plant material, such as, but not limited to, a tablet, capsule, a tea, powder, liquid, or other pharmacologically appropriate carrier. The composition may also be administered in a form without plant material, such as, but not limited to, a tablet, capsule, powder, liquid, tea, or other pharmacologically appropriate carrier. The composition should contain at least one of the group of three adrenergic amines N-methyl-β-phenethylamine; N,N-dimethylphenethylamine; and N-methyl-β-methylphenethylamine extracted from the plant material.

The Senegalia material used in accordance with the invention may consist of any portion of the plant, which contains useful amounts of the agents specified in this invention. For example, leaves of Senegalia Berlandieri are preferred to other parts of the plant, and may show levels of the related alkaloids of 5,000 ppm or more, based on dry matter, while the root or bark may contain no detectable amount of N-methyl-β-phenethylamine; N,N-dimethylphenethylamine; and N-methyl-β-methylphenethylamine and related alkaloids.

Though it is possible to use a variety of Senegalia materials in accordance with this invention, it is more convenient to utilize Senegalia materials which already exist in appropriate form and which are generally available as traditional herbs and remedies. For example, the agents present in the residues remaining after distillation of the leaves of Senegalia Berlandieri plant to obtain the desired alkaloids. In this respect, various species of the genus Senegalia from other geographical locations prepared in the same way are particularly useful.

The use of these substances and their combination with adrenergic amines will become readily apparent to those skilled in the art from the following detailed description of an embodiment of the present.

### DETAILED DESCRIPTION OF THE INVENTION

In one embodiment of the invention, material of the Senegalia plant species is given to humans orally, either concurrently with caloric restriction or in the absence of caloric restriction, for the purpose of controlling body weight. The Senegalia material so used is selected for its content of active agents as defined above such that the total amount of Senegalia material ingested provides a sufficient amount of the active agents to achieve the desired effects. In this respect, the preferred embodiment consists of a sufficient amount defined at least 0.025 mg of active agents per kilogram ideal body weight per dose at any one time.

Ingestion of active agents in the range of generally about 0.025 mg to generally about 2.5 mg per kilogram of ideal body weight per serving will be effective in accomplishing the desired goal of weight loss, though more preferred is a range of generally about 0.5 mg. to generally about 2 mg per kilogram of ideal body weight. Though ingestion of larger amounts of the agents will not diminish the beneficial effects. Still further, the preferred use of this invention is to administer single doses of from 25 to 250 mg up to 3 times daily, making a total daily dose of about 75 to 750 mg per day.

In this context, the active agents are deemed to be any one or more of N-methyl-β-phenethylamine; N,N-dimethylphenethylamine; and N-methyl-β-methylphenethylamine whereby the sufficient amount may be used by itself, or in combination of the agents that together provide a sufficient amount. In addition to these active agents, one or more other ingredients designed to modify the body's metabolism or increase energy are added.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In another embodiment, the oral dosage form includes a pharmaceutically acceptable carrier. As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well known in the art. Such may contain solubilizers, stabilizers, tonicity enhancing agents, buffers and preservatives.

In another embodiment the extract of the present invention may be administered in combination with one or more pharmacologically active compound, or an otherwise complementary ingredient(s) in therapeutically effective amounts (pharmaceutical combinations). For example, synergistic, or other complimentary effects can occur with central nervous system stimulants, such as amphetamines, amphetamine derivatives, caffeine, ephedrine, pseudoephedrine, and nicotine. In an embodiment, caffeine serves as a particularly effective combination with the composition of the present invention. Caffeine's main mechanism concerns antagonizing adenosine receptors. Adenosine causes sedation and relaxation when it acts upon its receptors, located in the brain. Caffeine prevents this action and causes alertness and wakefulness. By increasing catecholamine signaling (adrenaline and dopamine), caffeine can benefit mood and focus. Caffeine is also one of the most reliable and potent ways to temporarily increase strength through supplementation. Two distinct effects contribute to caffeine's fat-burning properties: a thermogenic effect (in the short term, caffeine increases heat production) and a weaker lipolytic effect (in the long term, caffeine causes triglycerides to release fatty acids, which the body can then use for fuel). More precisely, caffeine can increase the body's levels of cyclic adenosine monophosphate (cAMP). Elevated cAMP levels are associated with lower triglyceride levels in fat cells and improved protein synthesis in muscle cells.

This invention may be provided in other specific forms and embodiments without departing from the essential characteristics as described herein. The embodiments described above are to be considered in all aspects as illustrative only and not restrictive in any manner.

As described above, the present invention comprises a dietary supplement that increases energy and/or aids in weight loss. While particular embodiments of the invention have been described, it will be understood, however, that the invention is not limited thereto, since modifications may be made by those skilled in the art, particularly in light of the foregoing teachings. It is, therefore, contemplated that the claims cover any such modifications that incorporate those features or those improvements that embody the spirit and scope of the present invention.

What is claimed is:

1. A method for promoting energy and weight loss while stimulating thermogenesis and suppressing appetite which comprises administering to a human a composition comprising:
about 0.025 milligrams to about 2.5 milligrams per 1 kilogram of said human's body weight of an extract of one of the group consisting of Senegalia Berlandieri, or a pharmaceutically acceptable salt thereof.

2. The method of claim 1, where said composition is in an oral dosage form.

3. The method of claim 2, wherein said oral dosage form is selected from the group consisting of a chewable tablet, a quick dissolve tablet, an effervescent tablet, a hard gelatin capsule, a soft gelatin capsule, a powder, a reconstitutable powder, a suspension, an elixor, a caplet, a health bar, a liquid, a food and combinations thereof.

4. The method of claim 2, wherein said oral dosage is selected from the group consisting of immediate release, extended release, pulsed release, delayed release, timed release, variable release, controlled release and combinations thereof.

5. The method of claim 1, wherein said composition is administered once during a twenty-four hour period of time.

6. The method of claim 1, wherein said composition is administered at least twice during a twenty-four hour period of time.

7. A method for promoting weight loss while stimulating thermogenesis and suppressing appetite which comprises administering to a human a composition comprising:
about 25 milligrams to about 250 milligrams of an active ingredient selected from one of the group consisting of N-methyl-β-phenethylamine; N,N-dimethylphenethylamine; and N-methyl-β-methylphenethylamine;
wherein said active ingredient is extracted from Senegalia Berlandieri.

8. The method of claim 7, where said composition is in an oral dosage form.

9. The method of claim 8, wherein said oral dosage form is selected from the group consisting of a chewable tablet, a quick dissolve tablet, an effervescent tablet, a hard gelatin capsule, a soft gelatin capsule, a powder, a reconstitutable powder, a suspension, an elixor, a caplet, a health bar, a liquid, a food and combinations thereof.

10. The method of claim 8, wherein said oral dosage is selected from the group consisting of immediate release, extended release, pulsed release, delayed release, timed release, variable release, controlled release and combinations thereof.

11. The method of claim 7, wherein said composition is administered once during a twenty-four hour period of time.

12. The method of claim 7, wherein said composition is administered at least twice during a twenty-four hour period of time.

13. A pharmaceutical combination comprising an extract of one of the group consisting of Senegalia Berlandieri, or a pharmaceutically acceptable salt thereof and a stimulant.

14. A pharmaceutical combination according to claim 13 wherein said stimulant is caffeine.
